(19) United States
(12) Patent Application Publication
(10) Pub. No.: US 2012/0035204 A1
(43) Pub. Date: Feb. 9, 2012

(54) Methods for Treating Obesity and Disorders Associated with Hyperlipidemia in a Mammal

(76) Inventor: William J. Sasiela, (US)

(21) Appl. No.: 13/182,073

(22) Filed: Jul. 13, 2011

Related U.S. Application Data
(63) Continuation of application No. PCT/US2010/020999, filed on Jan. 14, 2010.
(60) Provisional application No. 61/144,544, filed on Jan. 14, 2009.

(51) Int. Cl. A61K 31/437; A61P 3/06; A61P 3/00; A61K 31/4468

(52) U.S. Cl. 514/292; 514/325

(57) ABSTRACT

The disclosure relates to methods for treating hyperlipidemia in a mammal. The present disclosure also relates to methods for treating and/or controlling obesity in a mammal. The methods involve combination therapies using a microsomal triglyceride transfer protein (MTP) inhibitor (for example, AEGR-733 and implitapide) and a DGAT inhibitor (for example, JTT-553 or PF-04415060). Co-administration of the MTP inhibitor with the DGAT inhibitor produces a therapeutic benefit, for example, a reduction in the concentration of cholesterol and/or triglycerides in the blood stream, but with fewer or reduced side effects than when higher dosages of the MTP inhibitor are used during monotherapy to provide the same or similar therapeutic benefit.

### REFERENCE TO RELATED APPLICATIONS

This application is a continuation of International Application No. PCT/US10/20999, filed Jan. 14, 2010, which claims priority to U.S. Provisional Application No. 61/144,544, filed Jan. 14, 2009, incorporated by reference herein in its entirety.

### BACKGROUND

Obesity and hyperlipidemia are major health concerns that can lead to a myriad of complications, including cardiovascular diseases, diabetes, and cancer. Obesity is often accompanied by hyperlipidemia, which is an elevation of lipids in the blood. These lipids include triglycerides, cholesterol, cholesterol esters, and phospholipids. Various epidemiological studies have demonstrated that lowering of total cholesterol (TC) and low density lipoprotein (LDL) cholesterol (LDL-C) is associated with a significant reduction of obesity and its associated health complications, for example, cardiovascular diseases. The National Cholesterol Education Program's (NCEP's) updated guidelines recommends that the overall goal for high-risk patients is to achieve less than 100 mg/dL of LDL, with a therapeutic option to set the goal for such patients to achieve less than 70 mg/dL of LDL.

The presence of elevated amounts of triglycerides in the blood is known as hypertriglyceridemia. Although triglycerides are necessary for good health, higher-than-normal triglyceride levels are often associated with increased risk of heart disease.

Another form of hyperlipidemia, known as hypercholesterolemia, is characterized by high cholesterol level, specifically very high LDL levels in the blood. High cholesterol levels can be successfully treated with medications and modifications in lifestyle. However, in some cases, as in familial hypercholesterolemia (FH), treatment can be challenging despite aggressive use of conventional therapy. FH is a serious genetic disorder due to homozygosity or compound heterozygosity for mutations in the LDL receptor. If left untreated, patients with homozygous FH (hoFH) develop atherosclerosis before the age 20 and generally do not survive past the age 30. However, patients diagnosed with hoFH are largely unresponsive to conventional drug therapy and have limited treatment options. Specifically, treatment with statins, which reduce LDL-C by inhibiting cholesterol synthesis and upregulating the hepatic LDL receptor, have negligible effect in hoFH patients whose LDL receptors are non-existent or defective. Therefore, there is a tremendous unmet medical need for new medical therapies for hoFH.

Microsomal triglyceride transfer protein (MTP) inhibitors have been shown clinically to lower plasma cholesterol levels. One exemplary MTP inhibitor is N-(2,2,2-Trifluorethyl)-9-[4-[4-[[[4′-(trifluoromethyl)[1,1′biphenyl]-2-Yl]carbonyl]amino]-1-piperidinyl]butyl]9H-fluorene-9-carboxamide (BMS-201038), developed by Bristol-Myers Squibb. See, U.S. Pat. Nos. 5,739,135; and 5,712,279. However, some patients treated with 25 mg/day of BMS-201038 experienced adverse events, for example, gastrointestinal disturbances, abnormalities in liver function, and hepatic steatosis. Another potent MTP inhibitor is known as implitapide. See, U.S. Pat. Nos. 6,265,431, 6,479,503, 5,952,498. During clinical studies, dosages of implitapide of 80 mg/day or greater, although therapeutically effective, were also found to result in certain adverse events, for example, gastrointestinal disturbances, abnormalities in liver function, and hepatic steatosis.

Accordingly, there is still a need for methods for aggressively treating hyperlipidemias and/or obesity that effectively lower, for example, circulating cholesterol and triglycerides levels but with fewer or reduced adverse effects that typically result when higher dosages of the MTP inhibitor are used alone in monotherapy.

### SUMMARY

The present disclosure provides methods for reducing the concentration of cholesterol and/or triglycerides in the blood of a mammal. The disclosure also provides methods for treating and/or controlling obesity in a patient in need thereof. The method includes administering a MTP inhibitor, such as AEGR-733 or implitapide, in combination with a DGAT (diacylglycerol acyltransferase) inhibitor, such as JTT-553 or PF-04415060. The MTP inhibitors can be administered at certain lower dosages that are still therapeutically effective when combined with a DGAT inhibitor but yet create fewer or reduced adverse effects when compared to therapies using therapeutically effective dosages of the MTP inhibitors during monotherapy. The administration of one or more MTP inhibitors, when administered in combination with one or more DGAT inhibitors, may provide an additive or synergistic therapeutic effect, e.g. may result in reduction of blood cholesterol and/or triglyceride levels that is greater than the sum of the expected cholesterol and/or triglyceride reduction due to administration of a MTP inhibitor and DGAT inhibitor when administered alone. In some embodiments, disclosed methods can result in fewer incidences of gastrointestinal or hepatic adverse events, e.g., hepatic steatosis, in a patient as compared to administration of a MTP inhibitor alone.

In one aspect, the disclosure provides a method of reducing the concentration of cholesterol and/or triglycerides in the blood of a mamma. The disclosure also provides a method of treating obesity in a mammal. The method comprises a combination therapy, which comprises administering to the mammal, for example, a human, a combination of DGAT inhibitor and AEGR-733, wherein AEGR-733 can be administered at about 2.5 mg/day to about 50 mg/day. In one embodiment, AEGR-733 is administered at a dosage of 10 mg/day. The DGAT inhibitor and AEGR-733 can be administered together in the same dosage form, or they may be administered in separate dosage forms. In the case of the separate dosage forms, the DGAT inhibitor can be administered before, after, or simultaneously with AEGR-733.

In another aspect, the disclosure provides a method of reducing the concentration of cholesterol and/or triglycerides in the blood of a mammal. The disclosure also provides a method of treating obesity in a mammal. The method comprises a combination therapy, which comprises administering to the mammal, for example, a human, a combination of DGAT inhibitor and implitapide, wherein implitapide is administered at about 20 mg/day to about 40 mg/day. The DGAT inhibitor and implitapide can be administered together in the same dosage form, or they may be administered in separate dosage forms. In the case of the separate dosage forms, the DGAT inhibitor can be administered before, after, or simultaneously with implitapide.

The present disclosure provides a method of reducing hepatic steatosis in a patient receiving a MTP inhibitor. The method comprises co-administering AEGR-733 and DGAT inhibitor to the patient. The AEGR-733 may be administered, for example, at a dosage of 2.5 mg/day to about 50 mg/day. In one embodiment, AEGR-733 is administered at about 10 mg/day. AEGR-733 and DGAT inhibitor may be administered together in the same dosage form or may be administered in separate dosage forms. In the case of the separate dosage forms, the DGAT inhibitor can be administered before, after, or simultaneously with AEGR-733.

In another aspect, the disclosure provides a method of reducing hepatic steatosis in a patient receiving a MTP inhibitor. The method comprises co-administering implitapide and DGAT inhibitor to the patient. The implitapide may be administered, for example, at a dosage of 2.5 mg/day to about 50 mg/day. In one embodiment, implitapide is administered at about 20 mg/day to about 40 mg/day. Implitapide and DGAT inhibitor may be administered together in the same dosage form or may be administered in separate dosage forms. In the case of the separate dosage forms, the DGAT inhibitor can be administered before, after, or simultaneously with implitapide.

In some embodiments, the amount of hepatic triglyceride in the patient's liver after one month is less than about 20% of the amount of hepatic triglyceride in a patient's liver if the MTP inhibitor is administered alone.

The foregoing methods can be used to treat (i) patients with hyperlipidemia, for example, hypercholesterolemia (for example, homozygous or heterozygous familial hypercholesterolemia) or hypertriglyceridemia, (ii) patients resistant to statin monotherapy, (iii) statin-intolerant patients, and/or (iv) patients having a combination of (i) and (ii), (i) and (iii), (ii) and (iii), and (i), (ii) and (iii).

### BRIEF DESCRIPTION OF THE DRAWINGS

FIG. 1 depicts a bar graph showing significantly reduced levels of hepatic triglyceride in DGAT−/− mice treated with AEGR-733 compared to DGAT+/− mice treated with AEGR-733.

FIG. 2 depicts a bar graph showing effect of DGAT activity on plasma lipid lowering effect of AEGR-733.

### DETAILED DESCRIPTION

This present disclosure relates to methods of reducing the concentration of cholesterol and/or triglycerides in the blood of a mammal. The disclosure also relates to methods of treating and/or reducing obesity in a patient in need thereof. The methods are based on combination therapies where a MTP inhibitor is administered with a DGAT inhibitor. In addition, the disclosure relates to a method of reducing hepatic steatosis induced by a MTP inhibitor by administering the MTP inhibitor together with a DGAT inhibitor.

For convenience, before further description, the meaning of certain terms and phrases used in the specification, examples, and appended claims are provided below.

### DEFINITIONS

The term “combination therapy,” as used herein, refers to co-administering a MTP inhibitor, for example, AEGR-733 and implitapide, or a combination thereof, and a DGAT inhibitor, for example, JTT-553 or PF-04415060, as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually weeks, months or years depending upon the combination selected). Combination therapy is intended to embrace administration of multiple therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single tablet or capsule having a fixed ratio of each therapeutic agent or in multiple, single capsules for each of the therapeutic agents. Sequential or substantially simultaneous administration of each therapeutic agent can be achieved by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection.

Combination therapy also can embrace the administration of the therapeutic agents as described above in further combination with other biologically active ingredients and non-drug therapies. Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

The components of the combination may be administered to a patient simultaneously or sequentially. It will be appreciated that the components may be present in the same pharmaceutically acceptable carrier and, therefore, are administered simultaneously. Alternatively, the active ingredients may be present in separate pharmaceutical carriers, such as, conventional oral dosage forms, that can be administered either simultaneously or sequentially.

The terms, “individual,” “patient,” or “subject” are used interchangeably herein and include any mammal, including animals, for example, primates, for example, humans, and other animals, for example, dogs, cats, swine, cattle, sheep, and horses. The compounds of the present disclosure can be administered to a mammal, such as a human, but can also be other mammals, for example, an animal in need of veterinary treatment, for example, domestic animals (for example, dogs, cats, and the like), farm animals (for example, cows, sheep, pigs, horses, and the like) and laboratory animals (for example, rats, mice, guinea pigs, and the like).

The phrase “minimizing adverse effects,” “reducing adverse events,” or “reduced adverse events,” as used herein refer to an amelioration or elimination of one or more undesired side effects associated with the use of MTP inhibitors of the present disclosure. Side effects of traditional use of the MTP inhibitors include, without limitation, nausea, diarrhea, gastrointestinal disorders, steatorrhea, abdominal cramping, distention, elevated liver function tests, fatty liver (hepatic steatosis); hepatic fat build up, polyneuropathy, peripheral neuropathy, rhabdomyolysis, arthralgia, myalgia, chest pain, rhinitis, dizziness, arthritis, peripheral edema, gastroenteritis, liver function tests abnormal, colitis, rectal hemorrhage, esophagitis, eructation, stomatitis, biliary pain, cheilitis, duodenal ulcer, dysphagia, enteritis, melena, gum hemorrhage, stomach ulcer, tenesmus, ulcerative stomatitis, hepatitis, pancreatitis, cholestatic jaundice, paresthesia, amnesia, libido decreased, emotional lability, incoordination, torticollis, facial paralysis, hyperkinesia, depression, hypesthesia, hypertonia, leg cramps, bursitis, tenosynovitis, myasthenia, tendinous contracture, myositis, hyperglycemia, creatine phosphokinase increased, gout, weight gain, hypoglycemia, anaphylaxis, angioneurotic edema, and bullous rashes (including erythema multiforme, Stevens-Johnson syndrome, and toxic epidermal necrolysis). Accordingly, the methods described herein provide an effective therapy while at the same time causing fewer or less significant adverse events.

In certain embodiments, side effects are partially eliminated. As used herein, the phrase “partially eliminated” refers to a reduction in the severity, extent, or duration of the particular side effect by at least 30%, 40%, 50%, 60%, 70%, 80%, 90%, 95% and 99% relative to that found by administering 25 mg/day of AEGR-733 during monotherapy or either 80 mg/day or 160 mg/day of implitapide during monotherapy. In certain embodiments, side effects are completely eliminated. Those skilled in the art are credited with the ability to detect and grade the severity, extent, or duration of side effects as well as the degree of amelioration of a side effect. For example, gastrointestinal side effects can be assessed, for example, using the Gastrointestinal Symptom Rating Scale. In some embodiments, two or more side effects are ameliorated.

The Gastrointestinal Symptom Rating Scale (“GSRS”) is an assessment tool for patients with general gastrointestinal complaints, and has been extensively validated in previous studies. The GSRS includes up to 15 items that addresses different gastrointestinal symptoms and typically uses a 7-point Likert response scale with verbal descriptors. The response scale is designed to measure the amount of discomfort a patient has experienced (none at all, minor, mild, moderate, moderately severe, severe, and very severe). A higher score in a GSRS cluster indicates more discomfort, with the scale from 1 (no discomfort) to 7. The recall period can refer, for example, to the past week. The 15 exemplary items can combine into five symptom clusters labeled reflux, abdominal pain, indigestion, diarrhea, and constipation. From individual items within a cluster, a mean score is calculated.

The term “synergistic” refers to two or more agents, e.g. a MTP inhibitor and a DGAT inhibitor, when taken together, produce a total joint effect that is greater than the sum of the effects of each drug when taken alone.

The term, “therapeutically effective” refers to the ability of an active ingredient, for example, AEGR-733 and implitapide, to elicit the biological or medical response that is being sought by a researcher, veterinarian, medical doctor or other clinician. Non-limiting examples include reduction of cholesterol (for example, LDL) and/or triglyceride levels in a patient, reduction of body mass in a patient, and the like.

The term, “therapeutically effective amount” includes the amount of an active ingredient, for example, AEGR-733 and implitapide, that will elicit the biological or medical response that is being sought by the researcher, veterinarian, medical doctor or other clinician. The compounds of the disclosure are administered in amounts effective at lowering the cholesterol concentration in the blood, and/or the triglyceride concentration in the blood. Alternatively, a therapeutically effective amount of an active ingredient is the quantity of the compound required to achieve a desired therapeutic and/or prophylactic effect, such as the amount of the active ingredient that results in the prevention of or a decrease in the symptoms associated with the condition (for example, to meet an end-point).

The terms, “pharmaceutically acceptable” or “pharmacologically acceptable” refer to molecular entities and compositions that do not produce an adverse, allergic or other untoward reaction when administered to an animal, or to a human, as appropriate. The term, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

Pharmaceutically acceptable salts of the disclosed compounds can be synthesized, for example, from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 20th ed., Lippincott Williams & Wilkins, Baltimore, Md., 2000, p. 704.

As used herein, the term “stereoisomers” refers to compounds made up of the same atoms bonded by the same bonds but having different spatial structures which are not interchangeable. The three-dimensional structures are called configurations. As used herein, the term “enantiomers” refers to two stereoisomers whose molecules are nonsuperimposable mirror images of one another. The terms “racemate,” “racemic mixture” or “racemic modification” refer to a mixture of equal parts of enantiomers.

### Methods

In general the present disclosure provides methods for reducing the concentration of cholesterol and/or triglycerides in the blood of a mammal. The disclosure also relates to methods of treating and/or reducing obesity in a patient in need thereof. The method comprises using one or more MTP inhibitors, for example, AEGR-733 or implitapide, in combination with a DGAT inhibitor, for example, JTT-553 or PF-04415060. The MTP inhibitors can be used at dosages lower than those already found to result in one or more adverse events, for example, gastrointestinal disorders, abnormalities in liver functional and/or hepatic steatosis (for example, 25 mg/day of AEGR-733, 80 mg/day of implitapide and 160 mg/day of implitapide have been found to cause gastrointestinal disorders, abnormalities in liver function and/or hepatic steatosis) but at doses which are therapeutically effective when combined with a DGAT inhibitor, for example, JTT-553 or PF-04415060. The dosages need not be smaller but may additionally and/or optionally be administered less frequently. It is contemplated that such a combination may be effective at reducing the concentration of cholesterol and/or triglycerides in the blood of a mammal even when larger dosages of AEGR-733 or implitapide are administered together with a dose of a DGAT inhibitor.

“Obesity” is a condition in which there is an excess of body fat. Typically, the definition of obesity is based on the Body Mass Index (BMI), which is calculated as body weight per height in meters squared (kg/m²). Obesity refers to a condition whereby an otherwise healthy patient has a Body Mass Index (BMI) greater than or equal to 30 kg/m² or a condition whereby a patient with at least one co-morbidity has a BMI greater than or equal to 27 kg/m². Obesity can also refer to those patients with a waist-to-hip ratio of 0.85 or more for women and 1.0 or more for men. Obesity can also refer to patients with a waist circumference of about 102 cm for males and about 88 cm for females.

A patient at risk of obesity is an otherwise healthy subject with a BMI of 25 kg/m² to less than 30 kg/m² or a subject with at least one co-morbidity with a BMI of 25 kg/m² to less than 27 kg/m². Alternatively or additionally, a patient at risk of obesity can refer to those patients with a waist-to-hip ratio of, e.g. 0.8 to 0.9 (women) and 0.9 to 1.0 (men). Such a patient may be in need of controlling obesity.

The increased risks associated with obesity occur at a lower Body Mass Index (BMI) in Asian patients or patients with Asian ancestry. In Asian countries, including Japan, obesity may refer to a condition whereby a patient with at least one obesity-induced or obesity-related co-morbidity, that requires weight reduction or that would be improved by weight reduction, has a BMI greater than or equal to 25 kg/m². For Asian patients a subject at risk of obesity is a subject with a BMI of greater than 23 kg/m² and less than 25 kg/m².

### Combination Therapies Using MTP Inhibitors and DGAT Inhibitors

The method comprises a combination therapy, which can be achieved by co-administering to the mammal a MTP inhibitor and a DGAT inhibitor. The MTP inhibitor and the DGAT inhibitor can be administered as a (i) single dosage form or composition, (ii) simultaneously as separate dosage forms or pharmaceutical compositions, (iii) sequentially, as separate dosage forms starting with the MTP inhibitor and then administering the DGAT inhibitor, or starting with the DGAT inhibitor and then administering the MTP inhibitor, (iv) successively, separated by for example 1-4 hours, 1-8 hours or 1-12 hours, a day, or 2 or more days, e.g. 2 to 3 days, or (v) individually followed by the combination. The methods disclosed herein may occur before, during, or after other dosing regimens that may include, for example MTP inhibitors, DGAT inhibitor, other agents for reducing cholesterol, such as statins, and/or agents for treating obesity such as, for example, a HMG-CoA reductase inhibitor, a bile acid sequestrant, a fibric acid derivative, niacin, squalene synthetase inhibitors, ACAT inhibitors, and/or CETP inhibitors. For example, the methods disclosed herein may occur after a patient has received statin monotherapy or statin combination therapy.

In some embodiments, a MTP inhibitor can be administered in escalating doses. Such escalating doses may comprise a first dose level and a second dose level. In other embodiments, escalating doses may comprise at least a first dosage level, a second dosage level, and a third dosage level, and optionally a fourth, fifth, or sixth dosage level. The DGAT inhibitor may be provided in one dosage level when in administered in combination with a MTP inhibitor, or may be administered in escalating doses.

A first, second, third or more dosage levels can be administered to a patient for about 2 days to about 6 months or more in duration. For example, first, second and/or third dose levels are each administered to a subject for about 1 week to about 26 weeks, or about 1 week to about 12 weeks, or about 1 week to about four weeks. Alternatively, the first, second and/or third dosage levels can be administered to a subject for about 2 days to about 40 days or to about 6 months.

The MTP inhibitor and/or the DGAT inhibitor each may be administered in a therapeutically effective amount and/or each in a synergistically effective amount. Such dosages of a MTP inhibitor and/or a DGAT inhibitor may, while not effective when used in monotherapy, may be effective when used in the combinations disclosed herein.

Administration of the MTP inhibitor and the DGAT inhibitor may result in fewer gastrointestinal or hepatic adverse events, such as hepatic steatosis, as compared to administration of a MTP inhibitor alone. In some embodiments, administration of the MTP inhibitor and the DGAT inhibitor may result in greater reduction of cholesterol and/or triglycerides in the blood and fewer gastrointestinal and/or hepatic adverse events as compared to administration of a MTP inhibitor or DGAT inhibitor alone. The level of cholesterol or triglycerides in the blood and reduction thereof, can be measured using conventional techniques known in the art, for example, a fasting blood test.

In certain other embodiments, the method produces an approximately 35%, 40% or more decrease in LDL-C in patients as compared to the patient's LDL-C level before treatment.

The methods disclosed herein may reduce or lower the concentration of serum cholesterol. It is understood that total serum cholesterol can be provided by very low density lipoproteins (VLDL), intermediate density lipoproteins (IDL), LDL and chylomicrons. Accordingly, it is contemplated that the combination therapies may reduce total blood cholesterol, or cholesterol provided by or associated with VLDL, IDL, LDL and chylomicrons. In addition, the methods disclosed herein may reduce or lower the concentration of serum triglycerides. It is understood that the serum triglycerides can be provided by VLDL and chylomicrons, and to a lesser extent by IDL and LDL. Accordingly, it is contemplated that the combination therapies may reduce triglycerides provided by or associated with VLDL, IDL, LDL and chylomicrons.

In other embodiments, the method produces a 2%, 3% or more reduction in body mass. For a patient with a BMI of greater than 30 kg/m², such a patient may have 3%, 3.5%, 5%, 6%, 7%, 8%, 9%, 10% or more reduction in body mass after, for example, one, two, four, eight, twelve, twenty-four, or more weeks of the disclosed therapy.

In another aspect, the present disclosure provides a method of reducing hepatic steatosis in a patient receiving MTP inhibitors. The method comprises co-administering a MTP inhibitor and a DGAT inhibitor to the patient. The MTP inhibitor may be administered, for example, at a dosage from 2.5 mg/day to about 50 mg/day. For example, MTP inhibitors may be administered at about 20 mg/day to about 40 mg/day. Higher doses may be appropriate for hoFH or severe refractory patients. MTP inhibitors and DGAT inhibitors may be administered together in the same dosage form or may be administered in separate dosage forms. In the case of separate dosage forms, DGAT inhibitor may be administered before, after, or simultaneously with, a MTP inhibitor.

Administration of the MTP inhibitor and the DGAT inhibitor may result in greater reduction of hepatic triglyceride in the patient's liver as compared to administration of a MTP inhibitor alone. For example administering to a patient a MTP inhibitor alone may cause an increase in hepatic fat from a baseline level while administering to a patient the MTP inhibitor and a DGAT inhibitor together may eliminate or lessen hepatic fat increase. In some embodiments, the amount of hepatic triglyceride in the patient's liver after one month is about 5%, 10%, 15%, 20%, 30%, or 40% less, e.g. about 5%-35% less, about 10%-30% less, or about 15%-25% less, than the amount of hepatic triglyceride in a patient's liver if the MTP inhibitor is administered alone.

### MTP Inhibitors

In one embodiment, the MTP inhibitor may be AEGR-733. As used herein, the phrase “BMS-201038” or “AEGR-733” refers to a compound known as N-(2,2,2-Trifluorethyl)-9-[4-[4-[[[4′-(trifluoromethyl)[1,1′biphenyl]-2-Yl]carbonyl]amino]-1-piperidinyl]butyl]9H-fluorene-9-carboxamide, having the formula:

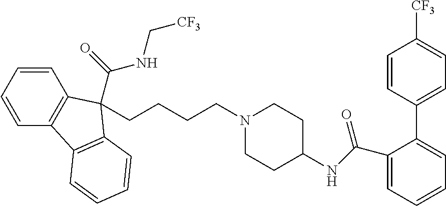

the piperidine N-oxide thereof, and stereoisomers, and pharmaceutically acceptable salts and esters thereof.

In another embodiment, the MTP inhibitor may include benzimidazole-based analogues of AEGR-733, for example, a compound having the formula shown below:

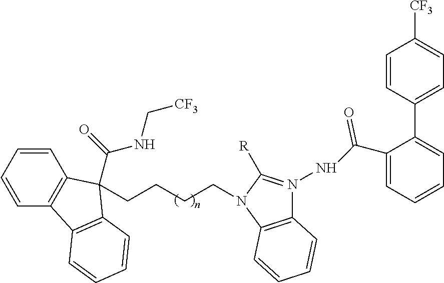

where n can be 0 to 10, and stereoisomers thereof, and pharmaceutically acceptable salts and esters thereof.

In another embodiment, the MTP inhibitor may be implitapide. As used herein, the phrase “implitapide” refers to a compound (2S)-2-cyclopentyl-2-[4-[(2,4-dimethyl-9H-pyrido[2,3-b]indol-9-yl)methyl]phenyl]-N-[(1S)-2-hydroxy-1-phenylethyl]ethanamide, and having the structure shown below:

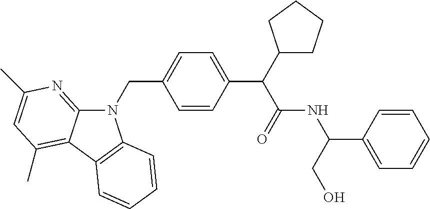

and stereoisomers thereof, and pharmaceutically acceptable salts and esters thereof.

Other MTP inhibitors include those developed by Surface Logix, Inc. e.g., SLx-4090.

### DGAT Inhibitors

As used herein, the term, “DGAT inhibitors” refers to compounds which inhibit diacylglycerol acyltransferase (DGAT) activity. DGAT is found in the microsomal fraction of cells. It catalyzes the final reaction in the glycerol phosphate pathway, considered to be the main pathway of triglyceride synthesis in cells by facilitating the joining of a diacylglycerol with a fatty acyl CoA, resulting in the formation of triglyceride.

Examples of DGAT inhibitor include, but are not limited to, JTT-553 and PF-04415060. In one embodiment, the DGAT inhibitor is JTT-553. In another embodiment, the DGAT inhibitor is PF-04415060. Other examples of DGAT inhibitors that may be used in the present disclosure include those described in U.S. Publication No. 20080064717 and WIPO Publication No. 2006/064189.

For example, a MTP inhibitor can be administered in combination with DGAT inhibitor. A DGAT inhibitor, such as JTT-553 or PF-04415060, may be co-administered at a dosage in the range of about 1 mg/day to about 1 g/day. Those skilled in the art will know the appropriate dosage of DGAT inhibitor to be co-administered with a MTP inhibitor to achieve therapeutic effectiveness while minimizing adverse effects.

### Therapies Using AEGR-733 and DGAT Inhibitor

In one aspect, the present disclosure provides a method of reducing the concentration of cholesterol and/or triglycerides in the blood of a mammal comprising administering a combination of DGAT inhibitor and AEGR-733 to a patient. In another aspect, the disclosure also provides a method of treating and/or controlling obesity in a patient.

Exemplary dosages for administration of AEGR-733 in combination with a DGAT inhibitor may include a dosage of about 2.5 mg/day to about 100 mg/day, e.g. 2.5 mg/day, 5 mg/day, 7.5 mg/day, 10 mg/day, 15 mg/day, 20 mg/day, 30 mg/day, or 50 mg/day or more of AEGR-733. In one embodiment, AEGR-733 is administered at about 10 mg/day. In some exemplary embodiments, the dosages of AEGR-733 may include lower dosages (e.g. about 2 to about 5 mg/day for one or more initial weeks of administration, and/or about 10 mg/day to about 50 mg/day for intermediate weeks of administration) but may be increased after such initial lower dosages up to doses of about 60 or 80 mg/day in combination with a DGAT inhibitor. For some patient populations, e.g. those patients with HoFH, dosage regimens that include higher doses of AEGR-733 (such as about 50 mg/day to about 90 mg/day) administered in a regimen that included a first week or weekly administration of lower dosages in combination with a DGAT inhibitor may be necessary or useful. Because administration of a DGAT inhibitor with higher dosages of AEGR-773 (or other MTP inhibitor) may e.g., reduce the amount of hepatic fat formed when AEGR-773 is administered alone, patients (such as those with HoFH) may successfully tolerate such higher doses, using, for example, a dose-escalation regimen

In an exemplary dose escalation regimen, the first dose level of AEGR-733 may be from about 2 to about 13 mg/day, and/or the second dose level may be about 5 to about 30 mg/day.

In an exemplary protocol, AEGR-733 initially is administered at a first dosage in the range of 2.5 to 7.5 mg/day for at least 4 weeks, is then administered at a second dosage in the range of 5 to 10 mg/day for at least 4 weeks, and is then administered at a third dosage in the range of 7.5 to 12.5 mg/day for at least 4 weeks. Such dosage regimens may each be in combination with, e.g., a DGAT inhibitor.

The first dosage of AEGR-733 can be for example 2.5 mg/day or 5 mg/day for about 4 weeks. The second dosage of AEGR-733 can be 7.5 mg/day for about 4 weeks. The third dosage of AEGR-733 can be 10 mg/day. In certain embodiments, the second dosage can be administered immediately following the first dosage, i.e., the second dosage is administered starting at five weeks from the initial first dosage. Similarly, in certain other embodiments, the third dosage of AEGR-733 can be administered immediately following the second dosage, e.g., the third dosage is administered at nine weeks from the initial first dosage.

Optionally, the method may include administering a second, third, or fourth dosage period of AEGR-733 alone, or in combination with a DGAT inhibitor. Such a fourth dosage may be in the range of 7.5-12.5 mg/day of AEGR-733 or more. A fourth dosage period may occur immediately after the second or third dosage, or may occur after a time interval, for example, a day, days, a week, or weeks after the third dosage. The fourth dosage may be administered to the subject for 1, 2, 3, 4 or more weeks.

### Therapies Using Implitapide and a DGAT Inhibitor

In one aspect, the disclosure provides a method of reducing the concentration of cholesterol and/or triglycerides in the blood of a mammal comprising administering a combination of DGAT inhibitor and implitapide to a patient.

Implitapide may be administered at a dosage in the range of 0.01 to 60 mg/day, or in the range of 20 to 60 mg/day, for example, 20 mg/day, 25 mg/day, 30 mg/day, 35 mg/day, 40 mg/day, 60 mg/day, or 80 mg/day or more. In one embodiment, implitapide can be administered at about 20 mg/day to about 40 mg/day. A DGAT inhibitor can be co-administered with implitipide at a dose of about, for example, 100 mg/day, 200 mg/day, 300 mg/day, 400 mg/day, 500 mg/day, 600 mg/day, 700 mg/day, 800 mg/day, 900 mg/day or 1 g/day.

### Formulation and Administration of the Active Ingredients

In certain embodiments, the MTP inhibitor (for example, AEGR-733 and implitapide) and the DGAT inhibitor can be administrated orally. For oral administration, the active ingredients may take the form of solid dose forms, for example, tablets (both swallowable and chewable forms), capsules or gelcaps, prepared by conventional means with pharmaceutically acceptable excipients and carriers such as binding agents (e.g. pregelatinised maize starch, polyvinylpyrrolidone, hydroxypropylmethylcellulose and the like), fillers (e.g. lactose, microcrystalline cellulose, calcium phosphate and the like), lubricants (e.g. magnesium stearate, talc, silica and the like), disintegrating agents (e.g. potato starch, sodium starch glycollate and the like), wetting agents (e.g. sodium laurylsulphate) and the like. Such tablets may also be coated by methods well known in the art.

Alternatively, it is contemplated that the active ingredients may be formulated for, and administered by, parenteral routes, for example, by intravenous routes, intramuscular routes, and by absorption through mucous membranes. It is contemplated that such formulations and parenteral modes of administration are known in the art.

The dosages described above may be administered in single or divided dosages of one to four times daily. The MTP inhibitor and DGAT inhibitor may be employed together in the same dosage form or in separate dosage forms taken at the same time, or at different times.

In certain embodiments, the methods disclosed herein, may minimize at least one of side effects associated with the administration of AEGR-733 and/or implitapide. Such side effects include, for example, nausea, diarrhea, gastrointestinal disorders, steatorrhea, abdominal cramping, distention, elevated liver function tests such as increases in liver enzymes such as alanine, minor fatty liver; hepatic fat build up, polyneuropathy, peripheral neuropathy, rhabdomyolysis, arthralgia, myalgia, chest pain, rhinitis, dizziness, arthritis, peripheral edema, gastroenteritis, liver function tests abnormal, colitis, rectal hemorrhage, esophagitis, eructation, stomatitis, biliary pain, cheilitis, duodenal ulcer, dysphagia, enteritis, melena, gum hemorrhage, stomach ulcer, tenesmus, ulcerative stomatitis, hepatitis, pancreatitis, cholestatic jaundice, paresthesia, amnesia, libido decreased, emotional lability, incoordination, torticollis, facial paralysis, hyperkinesia, depression, hypesthesia, hypertonia, leg cramps, bursitis, tenosynovitis, myasthenia, tendinous contracture, myositis, hyperglycemia, creatine phosphokinase increased, gout, weight gain, hypoglycemia, anaphylaxis, angioneurotic edema, and bullous rashes (including erythema multiforme, Stevens-Johnson syndrome, and toxic epidermal necrolysis). In some embodiments the minimization of the side effect is determined by assessing the grade, severity, extent, or duration by subject questionnaire.

### EXAMPLES

The examples that follow are intended in no way to limit the scope of this disclosure but are provided to illustrate the methods present disclosure. Many other embodiments of this disclosure will be apparent to one skilled in the art.

### Example 1

### Absence of DGAT1 Activity Enhances the Reduction of Hepatic Steatosis by AEGR-733

This study showed that the absence of DGAT1 activity enhanced the reduction of hepatic triglyceride levels by AEGR-733.

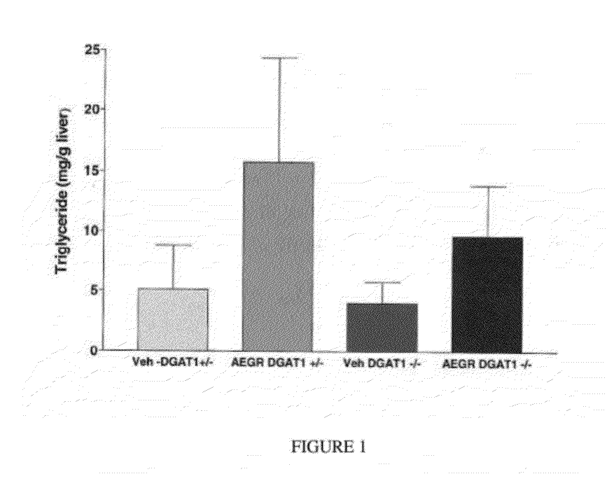

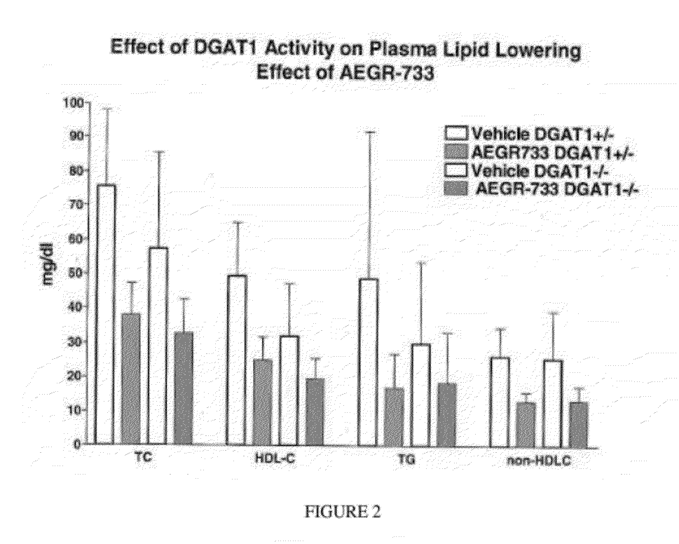

DGAT+/− mice or DGAT−/− mice (Jackson Laboratory, Maine, U.S.A.) were fed a chow diet ad libitum. Mice were dosed by oral gavage for four successive days with AEGR-733 or with vehicle control at about 9:00 AM. AEGR-733 was dissolved in M-pyrol and diluted to appropriate concentration in vehicle. Final vehicle composition was 10% M-pyrol, 80% water, 5% cremophore, and 5% ethanol. Dosing volume was 200 μl. The mice were euthanized approximately 6 hours after the last dose. Blood was obtained at time of sacrifice by orbital eye bleed and plasma was made for determination of total cholesterol (TC), high density lipoprotein cholesterol (HDL-C), triglyceride (TG) and non-HDL-C. FIG. 1 demonstrates that DGAT−/− mice treated with AEGR-733 exhibited significantly lower hepatic triglyceride levels compared to DGAT+/− mice treated with AEGR-733. FIG. 2 shows the effect of DGAT1 activity on the plasma lipid lowering effect of AEGR-733.

### REFERENCES

All publications and patents mentioned herein, including those items listed below, are hereby incorporated by reference in their entirety as if each individual publication are patent was specifically and individually incorporated by reference. In case of conflict, the present application, including any definitions herein, will control.

### EQUIVALENTS

It is understood that the disclosed disclosure is not limited to the particular methodology, protocols, and dosages described as these may vary. It is also to be understood that the terminology used herein is for the purpose of describing particular embodiments only, and is not intended to limit the scope of the present disclosure which will be limited only by the appended claims.

What is claimed is:

1. A method of reducing the concentration of cholesterol and/or triglycerides in the blood of a mammal in need thereof, comprising administering to the mammal a combination of a diacylglycerol acyltransferase (DGAT) inhibitor and a MTP inhibitor, wherein the method reduces the concentration of at least one of cholesterol or triglycerides in the blood but with a reduced incidence of an adverse event as compared to administration of the MTP inhibitor alone.

2. A method of treating and/or controlling obesity in a patient in need thereof, comprising administering to the patient a combination of a diacylglycerol acyltransferase (DGAT) inhibitor and a MTP inhibitor, wherein method has a reduced incidence of an adverse event as compared to administration of the MTP inhibitor alone.

3. The method of claim 1, wherein the MTP inhibitor is N-(2,2,2-Trifluorethyl)-9-[4-[4-[[[4′-(trifluoromethyl)[1,1′biphenyl]-2-Yl]carbonyl]amino]-1-piperidinyl]butyl]9H-fluorene-9-carboxamide or pharmaceutically acceptable salts thereof.

4. The method of claim 1, wherein the MTP inhibitor is implitapide or pharmaceutically acceptable salts thereof.

5. The method of claim 1, wherein the MTP inhibitor is administered at about 2.5 mg/day to about 50 mg/day.

6. The method of claim 5, wherein MTP inhibitor is administered at about 10 mg/day.

7. The method of claim 4, wherein the MTP inhibitor is administered at about 20 to 40 mg/day.

8. The method of claim 1, wherein the DGAT inhibitor and the compound are administered together in the same dosage form.

9. The method of claim 1, wherein the DGAT inhibitor and the compound are administered in separate dosage forms.

10. The method of claim 1, wherein the mammal is a human.

11. The method of claim 10, wherein the human has at least one of: hyperlipidemia, hypercholesterolemia, hypertriglyceridemia, or hyperchylomicronemia.

12. The method of claim 11, wherein the hypercholesterolemia is homozygous or heterozygous familial hypercholesterolemia.

13. The method of claim 1, wherein the DGAT inhibitor is JTT-553 or PF-04415060.

14. The method of claim 1, wherein the adverse event is hepatic steatosis.

15. A method of reducing the amount of hepatic triglyceride in a patient receiving a MTP inhibitor, comprising co-administering the MTP inhibitor and a DGAT inhibitor to the patient.

16. The method of claim 15, wherein the MTP inhibitor is N-(2,2,2-Trifluorethyl)-9-[4-[4-[[[4′-(trifluoromethyl)[1,1′biphenyl]-2-Yl]carbonyl]amino]-1-piperidinyl]butyl]9H-fluorene-9-carboxamide or pharmaceutically acceptable salts thereof.

17. The method of claim 15, wherein the MTP inhibitor is implitapide or pharmaceutically acceptable salts thereof.

18. The method of claim 16, wherein the MTP inhibitor is administered at about 2.5 mg/day to about 50 mg/day.

19. The method of claim 17, wherein the MTP inhibitor is administered at about 20 to 40 mg/day.

20. The method of claim 15, wherein the MTP inhibitor and DGAT inhibitor are administered at least daily.

21. The method of claim 15, wherein the amount of hepatic triglyceride in the patient's liver after one month is less than about 20% of the amount of hepatic triglyceride in a patient's liver if the MTP inhibitor is administered alone.
